(19) United States
(12) Patent Application Publication
(10) Pub. No.: US 2022/0049023 A1
(43) Pub. Date: Feb. 17, 2022

(54) NOVEL CRYSTALLINE FORMS OF SUGAMMADEX

(71) Applicants: Paolo AVALLE, Schachen (CH); Lorenzo CODAN, Schachen (CH); Patrick LARPENT, Schachen (CH); Jochen SCHÕLL, Schachen (CH); Werthenstein Biopharma GMBH, Schachen (CH)

(72) Inventors: Paolo Avalle, Luzern (CH); Lorenzo Codan, Olten (CH); Patrick Larpent, Luzern (CH); Jochen Scholl, Luzern (CH)

(73) Assignee: Werthenstein Biopharma GMBH, Schachen (CH)

(21) Appl. No.: 17/276,237

(22) PCT Filed: Sep. 25, 2019
(86) PCT No.: PCT/EP2019/075823

§ 371 Date: Mar. 15, 2021

Related U.S. Application Data
(60) Provisional application No. 62/737,140, filed on Sep. 27, 2018.

(51) Int. Cl. C08B 37/16

(52) U.S. Cl. CPC C08B 37/0012; C07B 2200/13

(57) ABSTRACT

The present invention provides a novel crystalline forms of sugammadex, designated herein as crystalline form Type 1 of sugammadex, crystalline form Type 2 of sugammadex, crystalline form Type 3 of sugammadex, crystalline form Type 8 of sugammadex, and crystalline form Type 9 of sugammadex, pharmaceutical compositions thereof, and methods for their use in the reversal of neuromuscular blockade induced by recuronium bromide or by vecuronium bromide in adults undergoing surgery.

### FIELD OF THE INVENTION

The present invention provides novel crystalline forms of sugammadex, designated herein as crystalline form Type 1 of sugammadex, crystalline form Type 2 of sugammadex, crystalline form Type 3 of sugammadex, crystalline form Type 8 of sugammadex, and crystalline form Type 9 of sugammadex, pharmaceutical compositions thereof, and methods for their use in the reversal of neuromuscular blockade induced by recuronium bromide and vecuronium bromide in adults undergoing surgery.

### BACKGROUND

Sugammadex is a modified cyclodextrin having the following structure:

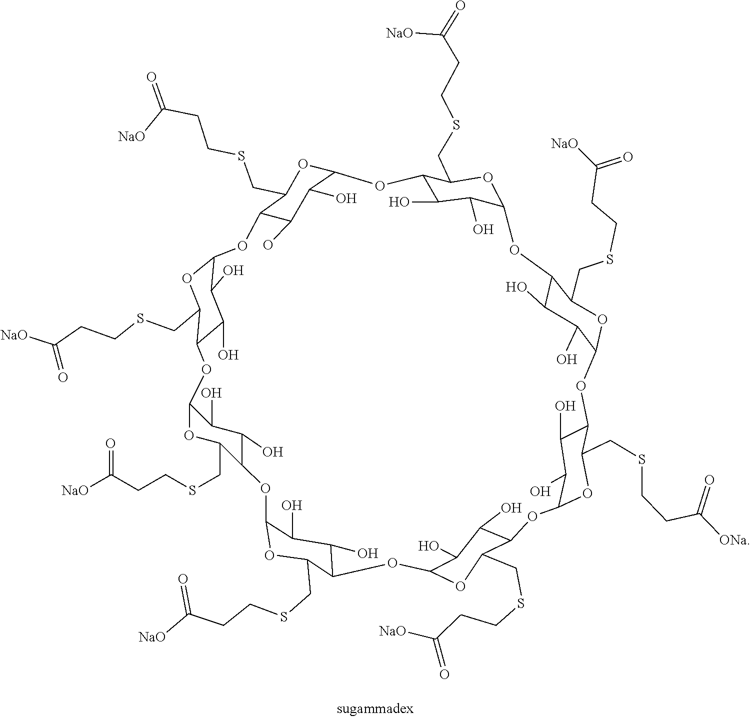

Sugammadex was approved in 2008 by the EMEA and in 2015 by the USFDA (and elsewhere) for the reversal of neuromuscular blockade induced by rocuronium bromide and vecuronium bromide in adults undergoing surgery. It is administered intravenously by injection in the form of a sterile solution under the brand name BRIDION®. Sugammadex is disclosed in WO2001/040316, published Jun. 7, 2001, together with a method for its synthesis. An improved synthesis of sugammadex is disclosed in U.S. Provisional Patent Application No. 62/681,889, filed Jun. 7, 2018. Other methods of producing sugammadex are also disclosed in the art. Once produced, the active ingredient is typically isolated as a wet cake and then dried under vacuum to obtain a powder meeting purity and residual solvent specifications. The powder is then dissolved in water for injection, the pH adjusted, and the resulting solution is filtered and filled into vials, sterilized and stored for use.

### SUMMARY OF THE INVENTION

In one aspect, the present invention provides novel crystalline forms of sugammadex. In one embodiment, there is provided crystalline form Type 1 of sugammadex. In another embodiment, there is provided crystalline form Type 2 of sugammadex. In another embodiment, there is provided crystalline form Type 3 of sugammadex. In another embodiment, there is provided crystalline form Type 8 of sugammadex. In another embodiment, there is provided crystalline form Type 9 of sugammadex.

In another aspect, the present invention provides methods for the use of each of the aforementioned crystalline forms of sugammadex in the preparation of a medicament for use in the reversal of neuromuscular blockade induced by rocuronium bromide and vecuronium bromide in adults undergoing surgery in accordance with its approved label.

### BRIEF DESCRIPTION OF THE DRAWINGS

FIG. 1 is a graph of a Powder X-Ray Diffraction (“PXRD”) pattern of sugammadex Type 1 crystalline form, generated using the equipment and methods described herein. The graph plots the intensity of the peaks as defined by counts per second versus the diffraction angle 2 theta (2Θ) in degrees.

FIG. 2 is a graph of a Powder X-Ray Diffraction (“PXRD”) pattern of sugammadex Type 2 crystalline form, generated using the equipment and methods described herein. The graph plots the intensity of the peaks as defined by counts per second versus the diffraction angle 2 theta (2Θ) in degrees.

FIG. 3 is a graph of a Powder X-Ray Diffraction (“PXRD”) pattern of sugammadex Type 3 crystalline form, generated using the equipment and methods described herein. The graph plots the intensity of the peaks as defined by counts per second versus the diffraction angle 2 theta (2Θ) in degrees.

FIG. 4 is a graph of a Powder X-Ray Diffraction (“PXRD”) pattern of sugammadex Type 8 crystalline form, generated using the equipment and methods described herein. The graph plots the intensity of the peaks as defined by counts per second versus the diffraction angle 2 theta (2Θ) in degrees.

FIG. 5 is a graph of a Powder X-Ray Diffraction (“PXRD”) pattern of sugammadex Type 9 crystalline form, generated using the equipment and methods described herein. The graph plots the intensity of the peaks as defined by counts per second versus the diffraction angle 2 theta (2Θ) in degrees.

### DETAILED DESCRIPTION OF THE INVENTION

### Definitions

The terms used herein have their ordinary meaning and the meaning of such terms is independent at each occurrence thereof. That notwithstanding and except where stated otherwise, the following definitions apply throughout the specification and claims.

° C. means degrees Celsius

FIG (or FIG. or Fig. or Fig or fig. or fig) means Figure (or figure) and refers to the corresponding figure

g means gram (or grams)

mL means milliliter (or milliliters)

“PXRD” is an abbreviation for powder x-ray diffraction

### Crystalline Forms of Sugammadex

The crystalline forms of sugammadex described herein may be prepared according to the procedures described below. For each procedure, starting quantities of sugammadex may be obtained from any suitable synthesis, including those described in PCT Publication No. WO2001/040316, Zhang, et al., published Jun. 7, 2001; and U.S. Provisional Patent Application No. 62/681,889, filed Jun. 7, 2018.

### PXRD

As those of ordinary skill in the art readily appreciate, the physical characteristics of a crystal may be effectively characterized by powder x-ray diffraction (PXRD) analysis. Such characterizations may be used to distinguish such crystals from other crystals. For each of the crystalline forms of sugammadex described herein, PXRD analysis was completed on a wet cake sample since drying the material leads to significant loss of crystallinity and to form change. PXRD data reported herein were acquired on a Bruker D8 Advance System configured in the Bragg-Brentano configuration and equipped with a Cu radiation source with monochromatization to Kα achieved using a nickel filter. A fixed slit optical configuration was employed for data acquisition. Data were acquired between 3 and 40° 2θ and a step size of 0.018. Samples were prepared by gently pressing the samples onto a shallow cavity zero background silicon holder. Wet cake samples were covered with Kapton® (polyimide film, DuPont, USA) foil in order to maintain the wet-sample-condition throughout data collection.

Those skilled in the art will recognize that the measurements of the PXRD peak locations for a given crystalline form of the same compound will vary within a margin of error. The margin of error for the 2-theta values measured as described herein is typically +/−0.2° 2θ. Variability can depend on such factors as the system, methodology, sample, and conditions used for measurement. As will also be appreciated by the skilled crystallographer, the intensities of the various peaks reported in the figures herein may vary due to a number of factors such as orientation effects of crystals in the x-ray beam, the purity of the material being analyzed, and/or the degree of crystallinity of the sample. The skilled crystallographer also will appreciate that measurements using a different wavelength will result in different shifts according to the Bragg-Brentano equation. Such further PXRD patterns generated by use of alternative wavelengths are considered to be alternative representations of the PXRD patterns of the crystalline material of the present invention and as such are within the scope of the present invention.

### Crystalline Form Type 1 of Sugammadex

Crystalline form Type 1 of sugammadex was prepared as follows:

1 g of sugammadex was added to 10 mL of a methanol/water mixture with a 10:1 ratio by volume at 25° C. and while applying magnetic stirring, resulting in a slurry. The slurry was kept at ambient temperature while stirring for 20 hours. A wet cake sample was produced by centrifuging an aliquot of the slurry to a wet paste. PXRD analysis of the wet cake produces the Type 1 pattern.

Physical characterization of crystalline form type 1 of sugammadex: PXRD

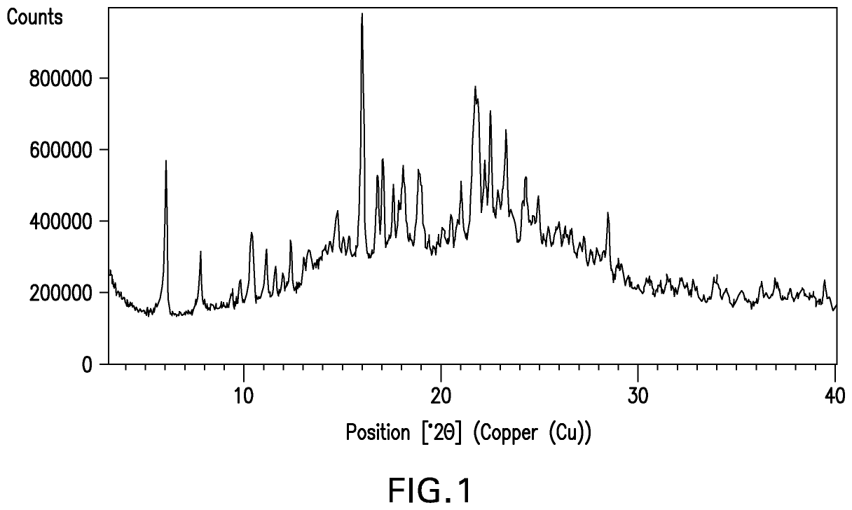

A PXRD pattern of crystalline form Type 1 of sugammadex generated using the equipment and procedures described above is displayed in FIG. 1. Thus, in another aspect, there is provided a crystalline form Type 1 of sugammadex characterized by a powder x-ray diffraction pattern substantially as shown in FIG. 1.

The intensity of the peaks (y-axis is in counts per second) were plotted versus the 2 theta angle (x-axis is in degrees 2 theta). In addition, the data were plotted with detector counts normalized for the collection time per step versus the 2 theta angle. Peak locations (on the 2 theta x-axis) consistent with these profiles are displaced in Table 1 (+/−0.2° 2 theta). The locations of these PXRD peaks are characteristic of the crystalline form Type 1 of sugammadex. Thus, in another aspect, crystalline form Type 1 of sugammadex is characterized by a powder x-ray diffraction pattern having each of the peak positions listed in Table 1, +/−0.2° 2-theta.

TABLE 1
Diffraction peaks and corresponding d-spacings for crystalline
form Type 1 of sugammadex

| Peak | Position [° Two | | | Diagnostic Peak |
|---|---|---|---|---|
| Number | Theta] | d-spacing [Å] | Rel. Int. [%] | Set |
| 1 | 6.04 | 14.62 | 80.73 | 1 |
| 2 | 7.80 | 11.33 | 21.01 | 2 |
| 3 | 9.39 | 9.41 | 5.71 | 3 |
| 4 | 9.83 | 8.99 | 5.95 | 3 |
| 5 | 10.42 | 8.49 | 24.97 | 2 |
| 6 | 11.14 | 7.94 | 17.79 | 3 |
| 7 | 11.57 | 7.64 | 9.20 | 3 |
| 8 | 11.99 | 7.38 | 7.31 | 3 |
| 9 | 12.36 | 7.15 | 17.56 | 3 |
| 10 | 13.33 | 6.64 | 10.91 | 3 |
| 11 | 14.09 | 6.28 | 7.86 | 3 |
| 12 | 14.40 | 6.14 | 9.22 | 3 |
| 13 | 14.72 | 6.01 | 20.89 | 2 |
| 14 | 15.05 | 5.88 | 6.45 | 3 |
| 15 | 15.31 | 5.78 | 6.09 | 3 |
| 16 | 16.01 | 5.53 | 100.00 | 1 |
| 17 | 16.79 | 5.28 | 34.80 | 2 |
| 18 | 17.05 | 5.20 | 42.04 | 1 |
| 19 | 17.35 | 5.11 | 9.81 | 3 |
| 20 | 17.59 | 5.04 | 28.37 | 2 |
| 21 | 17.86 | 4.96 | 20.45 | 2 |
| 22 | 18.12 | 4.89 | 39.57 | 2 |
| 23 | 18.42 | 4.81 | 7.27 | 3 |
| 24 | 18.91 | 4.69 | 36.34 | 2 |
| 25 | 20.09 | 4.42 | 10.02 | 3 |
| 26 | 20.30 | 4.37 | 5.05 | 3 |
| 27 | 20.51 | 4.33 | 14.43 | 3 |
| 28 | 21.01 | 4.22 | 26.20 | 2 |
| 29 | 21.83 | 4.07 | 72.54 | 1 |
| 30 | 22.24 | 3.99 | 36.33 | 2 |
| 31 | 22.50 | 3.95 | 59.81 | 1 |
| 32 | 22.90 | 3.88 | 25.72 | 2 |
| 33 | 23.31 | 3.81 | 49.28 | 1 |
| 34 | 23.55 | 3.77 | 15.77 | 3 |
| 35 | 24.30 | 3.66 | 29.97 | 2 |
| 36 | 24.73 | 3.60 | 13.46 | 3 |
| 37 | 24.92 | 3.57 | 26.36 | 2 |
| 38 | 25.43 | 3.50 | 6.77 | 3 |
| 39 | 25.81 | 3.45 | 9.19 | 3 |
| 40 | 25.98 | 3.43 | 10.81 | 3 |
| 41 | 26.30 | 3.39 | 10.55 | 3 |
| 42 | 26.61 | 3.35 | 8.86 | 3 |
| 43 | 27.05 | 3.29 | 6.65 | 3 |
| 44 | 27.25 | 3.27 | 11.51 | 3 |
| 45 | 27.64 | 3.22 | 9.84 | 3 |
| 46 | 27.95 | 3.19 | 14.63 | 3 |
| 47 | 28.47 | 3.13 | 34.24 | 2 |
| 48 | 28.95 | 3.08 | 11.40 | 3 |
| 49 | 29.17 | 3.06 | 10.44 | 3 |
| 50 | 29.53 | 3.02 | 5.97 | 3 |
| 51 | 29.76 | 3.00 | 5.33 | 3 |
| 52 | 29.94 | 2.98 | 7.50 | 3 |

In a further aspect, the PXRD peak locations displayed in Table 1 and/or FIG. 1 most characteristic of crystalline form Type 1 of sugammadex can be selected and grouped as “diagnostic peak sets” to conveniently distinguish this crystalline form from others. Selections of such characteristic peaks are set out in Table 1 in the column labeled Diagnostic Peak Set.

Thus, in another aspect, there is provided a crystalline form Type 1 of sugammadex characterized by a powder x-ray diffraction pattern comprising each of the 2-theta values listed in Diagnostic Peak Set 1 in Table 1, +/−0.2° 2-theta.

In another aspect, there is provided a crystalline form Type 1 of sugammadex characterized by a powder x-ray diffraction pattern comprising each of the 2-theta values listed in Diagnostic Peak Set 2 in Table 1, +/−0.2° 2-theta.

In another aspect, there is provided a crystalline form Type 1 of sugammadex characterized by a powder x-ray diffraction pattern comprising each of the 2-theta values listed in Diagnostic Peak Set 3 in Table 1, +/−0.2° 2-theta.

In another aspect, there is provided a crystalline form Type 1 of sugammadex characterized by a powder x-ray diffraction pattern comprising each of the 2-theta values listed in Diagnostic Peak Set 1 and Diagnostic Peak Set 2 in Table 1, +/−0.2° 2-theta.

In another aspect, there is provided a crystalline form Type 1 of sugammadex characterized by a powder x-ray diffraction pattern comprising each of the 2-theta values listed in Diagnostic Peak Set 1 and Diagnostic Peak Set 3 in Table 1, +/−0.2° 2-theta.

In another aspect, there is provided a crystalline form Type 1 of sugammadex characterized by a powder x-ray diffraction pattern comprising each of the 2-theta values listed in Diagnostic Peak Set 1 and Diagnostic Peak Set 2 and Diagnostic Peak Set 3 in Table 1, +/−0.2° 2-theta.

Physical characterization of crystalline form type 1 of Sugammadex: NMR

### Crystalline Form Type 2 of Sugammadex

Crystalline form Type 2 of sugammadex was prepared as follows:

500 mg of sugammadex was added to 5 mL of a methanol/water mixture with a 5/1 ratio by volume at 40° C. and while applying magnetic stirring, resulting in a slurry. The slurry was kept at 40° C. while stirring for 20 hours. A wet cake sample was produced by centrifuging an aliquot of the slurry to a wet paste. PXRD analysis of the wet cake produces the Type 2 pattern.

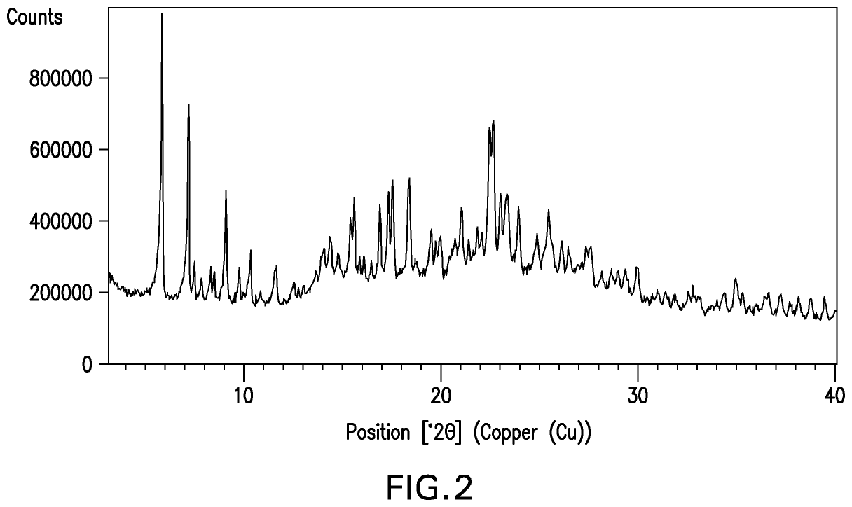

A PXRD pattern of crystalline form Type 2 of sugammadex generated using the equipment and procedures described above is displayed in FIG. 2. Thus, in another aspect, there is provided a crystalline form Type 2 of sugammadex characterized by a powder x-ray diffraction pattern substantially as shown in FIG. 2.

The intensity of the peaks (y-axis is in counts per second) were plotted versus the 2 theta angle (x-axis is in degrees 2 theta). In addition, the data were plotted with detector counts normalized for the collection time per step versus the 2 theta angle. Peak locations (on the 2 theta x-axis) consistent with these profiles are displaced in Table 2 (+/−0.2° 2 theta). The locations of these PXRD peaks are characteristic of the crystalline form Type 2 of sugammadex. Thus, in another aspect, crystalline form Type 2 of sugammadex is characterized by a powder x-ray diffraction pattern having each of the peak positions listed in Table 2, +/−0.2° 2-theta.

TABLE 2
Diffraction peaks and corresponding d-spacings for crystalline
form Type 2 of sugammadex

| | | | | Preferred |
|---|---|---|---|---|
| Peak | Pos. [° Two | | | Diagnostic Peak |
| Number | Theta] | d-spacing [Å] | Rel. Int. [%] | Set |
| 1 | 5.72 | 15.44 | 100.00 | 1 |
| 2 | 6.94 | 12.72 | 16.87 | 3 |
| 3 | 7.07 | 12.50 | 72.39 | 1 |
| 4 | 7.37 | 11.98 | 15.38 | 3 |
| 5 | 7.73 | 11.42 | 8.39 | 3 |
| 6 | 8.18 | 10.79 | 11.52 | 3 |
| 7 | 8.38 | 10.54 | 10.17 | 3 |
| 8 | 8.98 | 9.84 | 43.20 | 1 |
| 9 | 9.64 | 9.17 | 11.62 | 3 |
| 10 | 10.13 | 8.73 | 10.01 | 3 |
| 11 | 10.22 | 8.65 | 17.10 | 3 |
| 12 | 11.44 | 7.73 | 13.33 | 3 |
| 13 | 11.51 | 7.68 | 13.92 | 3 |
| 14 | 12.41 | 7.12 | 5.81 | 3 |
| 15 | 13.55 | 6.53 | 5.39 | 3 |
| 16 | 13.82 | 6.40 | 10.61 | 3 |
| 17 | 13.93 | 6.35 | 12.82 | 3 |
| 18 | 14.29 | 6.19 | 16.86 | 3 |
| 19 | 14.68 | 6.03 | 10.13 | 3 |
| 20 | 15.31 | 5.78 | 22.31 | 2 |
| 21 | 15.49 | 5.71 | 31.26 | 2 |
| 22 | 15.76 | 5.62 | 6.12 | 3 |
| 23 | 15.98 | 5.54 | 7.93 | 3 |
| 24 | 16.37 | 5.41 | 5.38 | 3 |
| 25 | 16.81 | 5.27 | 29.87 | 2 |
| 26 | 17.22 | 5.15 | 32.43 | 2 |
| 27 | 17.44 | 5.08 | 39.45 | 1 |
| 28 | 18.28 | 4.85 | 40.64 | 1 |
| 29 | 18.59 | 4.77 | 7.03 | 3 |
| 30 | 18.70 | 4.74 | 5.23 | 3 |
| 31 | 19.40 | 4.57 | 18.06 | 3 |
| 32 | 19.63 | 4.52 | 14.27 | 3 |
| 33 | 19.83 | 4.47 | 15.07 | 3 |
| 34 | 20.32 | 4.37 | 7.71 | 3 |
| 35 | 20.46 | 4.34 | 9.95 | 3 |
| 36 | 20.62 | 4.30 | 14.42 | 3 |
| 37 | 20.95 | 4.24 | 26.09 | 2 |
| 38 | 21.31 | 4.17 | 14.16 | 3 |
| 39 | 21.54 | 4.12 | 8.45 | 3 |
| 40 | 21.74 | 4.08 | 18.17 | 3 |
| 41 | 21.97 | 4.04 | 14.23 | 3 |
| 42 | 22.37 | 3.97 | 57.27 | 1 |
| 43 | 22.55 | 3.94 | 60.42 | 1 |
| 44 | 22.91 | 3.88 | 30.50 | 2 |
| 45 | 23.27 | 3.82 | 31.14 | 2 |
| 46 | 23.56 | 3.77 | 5.30 | 3 |
| 47 | 23.84 | 3.73 | 25.95 | 2 |
| 48 | 24.78 | 3.59 | 15.91 | 3 |
| 49 | 25.37 | 3.51 | 25.78 | 2 |
| 50 | 26.04 | 3.42 | 14.06 | 3 |
| 51 | 26.38 | 3.38 | 9.99 | 3 |
| 52 | 26.87 | 3.32 | 5.97 | 3 |
| 53 | 27.07 | 3.29 | 8.27 | 3 |
| 54 | 27.34 | 3.26 | 15.57 | 3 |
| 55 | 27.52 | 3.24 | 19.28 | 3 |
| 56 | 28.07 | 3.18 | 18.76 | 3 |
| 57 | 28.29 | 3.15 | 17.15 | 3 |
| 58 | 28.56 | 3.12 | 25.82 | 2 |
| 59 | 28.88 | 3.09 | 27.45 | 2 |
| 60 | 29.26 | 3.05 | 23.13 | 2 |
| 61 | 29.86 | 2.99 | 12.43 | 3 |

In a further aspect, the PXRD peak locations displayed in Table 2 and/or FIG. 2 most characteristic of crystalline form Type 2 of sugammadex can be selected and grouped as “diagnostic peak sets” to conveniently distinguish this crystalline form from others. Selections of such characteristic peaks are set out in Table 2 in the column labeled Diagnostic Peak Set.

Thus, in another aspect, there is provided a crystalline form Type 2 of sugammadex characterized by a powder x-ray diffraction pattern comprising each of the 2-theta values listed in Diagnostic Peak Set 1 in Table 2, +/−0.2° 2-theta.

In another aspect, there is provided a crystalline form Type 2 of sugammadex characterized by a powder x-ray diffraction pattern comprising each of the 2-theta values listed in Diagnostic Peak Set 2 in Table 2, +/−0.2° 2-theta.

In another aspect, there is provided a crystalline form Type 2 of sugammadex characterized by a powder x-ray diffraction pattern comprising each of the 2-theta values listed in Diagnostic Peak Set 3 in Table 2, +/−0.2° 2-theta.

In another aspect, there is provided a crystalline form Type 2 of sugammadex characterized by a powder x-ray diffraction pattern comprising each of the 2-theta values listed in Diagnostic Peak Set 1 and Diagnostic Peak Set 2 in Table 2, +/−0.2° 2-theta.

In another aspect, there is provided a crystalline form Type 2 of sugammadex characterized by a powder x-ray diffraction pattern comprising each of the 2-theta values listed in Diagnostic Peak Set 1 and Diagnostic Peak Set 2 and Diagnostic Peak Set 3 in Table 2, +/−0.2° 2-theta.

Physical characterization of crystalline form Type 2 of sugammadex: NMR

### Crystalline Form TYPE 3 of Sugammadex

Crystalline form Type 3 of sugammadex was prepared as follows:

1 g of sugammadex was added to 10 mL of a methanol/water mixture with a 10/1 ratio by volume at 40° C. and while applying magnetic stirring, resulting in a slurry. The slurry was kept at 40° C. while stirring for 3 days. A wet cake sample was produced by centrifuging an aliquot of the slurry to a wet paste. PXRD analysis of the wet cake produces the Type 3 pattern.

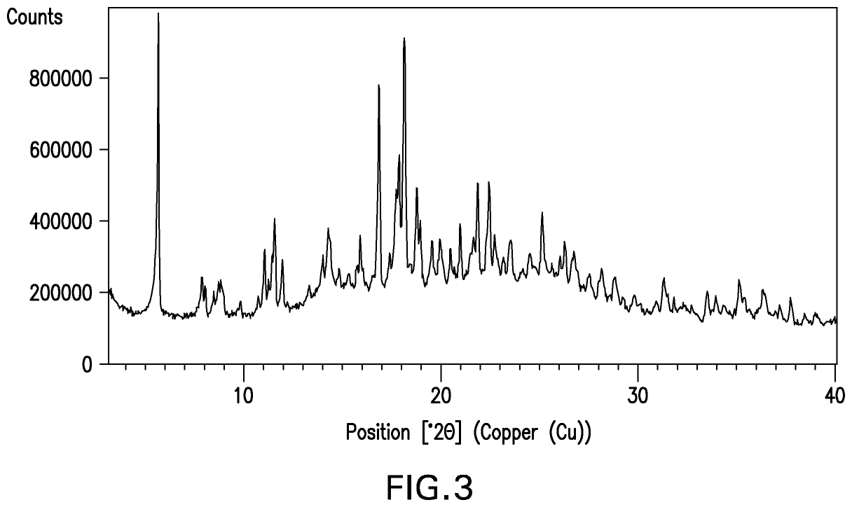

A PXRD pattern of crystalline form Type 3 of sugammadex generated using the equipment and procedures described above is displayed in FIG. 3. Thus, in another aspect, there is provided a crystalline form Type 3 of sugammadex characterized by a powder x-ray diffraction pattern substantially as shown in FIG. 3.

The intensity of the peaks (y-axis is in counts per second) were plotted versus the 2 theta angle (x-axis is in degrees 2 theta). In addition, the data were plotted with detector counts normalized for the collection time per step versus the 2 theta angle. Peak locations (on the 2 theta x-axis) consistent with these profiles are displaced in Table 3 (+/−0.2° 2 theta). The locations of these PXRD peaks are characteristic of the crystalline form Type 3 of sugammadex. Thus, in another aspect, crystalline form Type 3 of sugammadex is characterized by a powder x-ray diffraction pattern having each of the peak positions listed in Table 3, +/−0.2° 2-theta.

TABLE 3
Diffraction peaks and corresponding d-spacings for crystalline
form Type 3 of sugammadex

| | | | | Preferred |
|---|---|---|---|---|
| | | | | Diagnostic |
| | Pos. [° Two | | | Peak |
| Peak Number | Theta] | d-spacing [Å] | Rel. Int. [%] | Set |
| 1 | 5.60 | 15.76 | 100.00 | 1 |
| 2 | 7.75 | 11.40 | 10.02 | 3 |
| 3 | 7.89 | 11.20 | 10.14 | 3 |
| 4 | 7.96 | 11.10 | 10.66 | 3 |
| 5 | 8.41 | 10.50 | 8.89 | 3 |
| 6 | 8.72 | 10.13 | 13.97 | 3 |
| 7 | 8.84 | 9.99 | 10.78 | 3 |
| 8 | 11.13 | 7.94 | 11.20 | 3 |
| 9 | 11.47 | 7.71 | 32.26 | 2 |
| 10 | 11.83 | 7.47 | 12.28 | 3 |
| 11 | 13.99 | 6.32 | 10.98 | 3 |
| 12 | 14.23 | 6.22 | 23.46 | 2 |
| 13 | 15.83 | 5.59 | 14.46 | 3 |
| 14 | 16.80 | 5.27 | 65.04 | 1 |
| 15 | 17.36 | 5.10 | 11.91 | 3 |
| 16 | 17.81 | 4.98 | 44.50 | 1 |
| 17 | 18.10 | 4.90 | 87.11 | 1 |
| 18 | 18.38 | 4.82 | 7.77 | 3 |
| 19 | 18.71 | 4.74 | 29.15 | 2 |
| 20 | 18.87 | 4.70 | 19.36 | 3 |
| 21 | 19.91 | 4.45 | 11.11 | 3 |
| 22 | 20.44 | 4.34 | 6.97 | 3 |
| 23 | 20.92 | 4.24 | 15.60 | 3 |
| 24 | 21.57 | 4.12 | 16.09 | 3 |
| 25 | 21.82 | 4.07 | 32.05 | 2 |
| 26 | 22.38 | 3.97 | 38.87 | 1 |
| 27 | 22.58 | 3.93 | 15.37 | 3 |
| 28 | 22.74 | 3.91 | 20.74 | 2 |
| 29 | 22.96 | 3.87 | 11.66 | 3 |
| 30 | 23.12 | 3.84 | 16.74 | 3 |
| 31 | 23.48 | 3.79 | 25.11 | 2 |
| 32 | 23.82 | 3.73 | 11.67 | 3 |
| 33 | 24.04 | 3.70 | 15.14 | 3 |
| 34 | 24.14 | 3.68 | 14.29 | 3 |
| 35 | 24.45 | 3.64 | 21.53 | 2 |
| 36 | 24.63 | 3.61 | 16.48 | 3 |
| 37 | 24.77 | 3.59 | 16.39 | 3 |
| 38 | 25.15 | 3.54 | 28.07 | 2 |
| 39 | 25.37 | 3.51 | 15.05 | 3 |
| 40 | 25.58 | 3.48 | 15.09 | 3 |
| 41 | 25.78 | 3.45 | 11.74 | 3 |
| 42 | 25.98 | 3.43 | 16.27 | 3 |
| 43 | 26.23 | 3.39 | 21.49 | 2 |
| 44 | 26.70 | 3.34 | 17.18 | 3 |
| 45 | 27.17 | 3.28 | 5.76 | 3 |
| 46 | 27.46 | 3.25 | 10.00 | 3 |
| 47 | 28.10 | 3.17 | 13.47 | 3 |
| 48 | 28.79 | 3.10 | 13.02 | 3 |
| 49 | 29.24 | 3.05 | 8.12 | 3 |
| 50 | 29.76 | 3.00 | 12.42 | 3 |
| 51 | 30.12 | 2.96 | 11.68 | 3 |
| 52 | 30.89 | 2.89 | 18.21 | 3 |
| 53 | 31.27 | 2.86 | 29.05 | 2 |
| 54 | 31.43 | 2.84 | 23.20 | 2 |
| 55 | 31.79 | 2.81 | 21.42 | 2 |
| 56 | 32.08 | 2.79 | 19.01 | 3 |
| 57 | 32.28 | 2.77 | 19.86 | 3 |
| 58 | 32.68 | 2.74 | 18.11 | 3 |
| 59 | 33.00 | 2.71 | 15.27 | 3 |
| 60 | 33.49 | 2.67 | 24.90 | 2 |
| 61 | 33.94 | 2.64 | 26.71 | 2 |
| 62 | 34.33 | 2.61 | 26.76 | 2 |

In a further aspect, the PXRD peak locations displayed in Table 3 and/or FIG. 3 most characteristic of crystalline form Type 3 of sugammadex can be selected and grouped as “diagnostic peak sets” to conveniently distinguish this crystalline form from others. Selections of such characteristic peaks are set out in Table 3 in the column labeled Diagnostic Peak Set.

Thus, in another aspect, there is provided a crystalline form Type 3 of sugammadex characterized by a powder x-ray diffraction pattern comprising each of the 2-theta values listed in Diagnostic Peak Set 1 in Table 3, +/−0.2° 2-theta.

In another aspect, there is provided a crystalline form Type 3 of sugammadex characterized by a powder x-ray diffraction pattern comprising each of the 2-theta values listed in Diagnostic Peak Set 2 in Table 3, +/−0.2° 2-theta.

In another aspect, there is provided a crystalline form Type 3 of sugammadex characterized by a powder x-ray diffraction pattern comprising each of the 2-theta values listed in Diagnostic Peak Set 3 in Table 3, +/−0.2° 2-theta.

In another aspect, there is provided a crystalline form Type 3 of sugammadex characterized by a powder x-ray diffraction pattern comprising each of the 2-theta values listed in Diagnostic Peak Set 1 and Diagnostic Peak Set 2 in Table 3, +/−0.2° 2-theta.

In another aspect, there is provided a crystalline form Type 3 of sugammadex characterized by a powder x-ray diffraction pattern comprising each of the 2-theta values listed in Diagnostic Peak Set 1 and Diagnostic Peak Set 2 and Diagnostic Peak Set 3 in Table 3, +/−0.2° 2-theta.

Physical characterization of crystalline form Type 3 of sugammadex: NMR

### Crystalline Form Type 8 of Sugammadex

Crystalline form Type 8 of sugammadex was prepared as follows:

0.5 g of sugammadex was dissolved in 1.5 mL of water at 25° C. while applying magnetic stirring, resulting in a clear solution. Subsequently, 6 mL of methanol were added over a 5-minute time period while applying slow magnetic stirring, resulting in the precipitation of a solid. The slurry was stirred for another 1 hour at 25° C. A wet cake sample was produced by centrifuging an aliquot of the slurry to a wet paste.

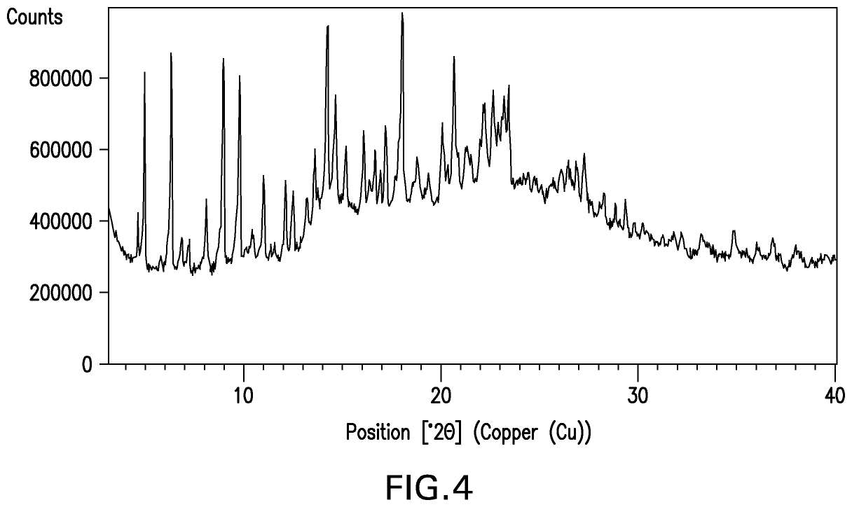

A PXRD pattern of crystalline form Type 8 of sugammadex generated using the equipment and procedures described above is displayed in FIG. 4. Thus, in another aspect, there is provided a crystalline form Type 8 of sugammadex characterized by a powder x-ray diffraction pattern substantially as shown in FIG. 4.

The intensity of the peaks (y-axis is in counts per second) were plotted versus the 2 theta angle (x-axis is in degrees 2 theta). In addition, the data were plotted with detector counts normalized for the collection time per step versus the 2 theta angle. Peak locations (on the 2 theta x-axis) consistent with these profiles are displaced in Table 4 (+/−0.2° 2 theta). The locations of these PXRD peaks are characteristic of the crystalline form Type 8 of sugammadex. Thus, in another aspect, crystalline form Type 8 of sugammadex is characterized by a powder x-ray diffraction pattern having each of the peak positions listed in Table 4, +/−0.2° 2-theta.

TABLE 4
Diffraction peaks and corresponding d-spacings for crystalline
form Type 8 of sugammadex

| | | | | Preferred |
|---|---|---|---|---|
| Peak | Pos. [° Two | | | Diagnostic Peak |
| Number | Theta] | d-spacing [Å] | Rel. Int. [%] | Set |
| 1 | 4.48 | 19.71 | 17.77 | 3 |
| 2 | 4.82 | 18.32 | 69.15 | 2 |
| 3 | 6.19 | 14.27 | 94.88 | 1 |
| 4 | 6.73 | 13.13 | 16.64 | 3 |
| 5 | 7.09 | 12.46 | 14.56 | 3 |
| 6 | 7.97 | 11.08 | 33.06 | 3 |
| 7 | 8.85 | 9.98 | 100.00 | 1 |
| 8 | 9.54 | 9.27 | 26.65 | 3 |
| 9 | 9.66 | 9.15 | 83.64 | 1 |
| 10 | 10.27 | 8.60 | 11.82 | 3 |
| 11 | 10.36 | 8.53 | 12.06 | 3 |
| 12 | 10.89 | 8.12 | 44.45 | 2 |
| 13 | 12.00 | 7.37 | 37.73 | 2 |
| 14 | 12.38 | 7.14 | 29.87 | 3 |
| 15 | 13.08 | 6.76 | 17.53 | 3 |
| 16 | 13.48 | 6.56 | 36.51 | 2 |
| 17 | 13.64 | 6.49 | 13.79 | 3 |
| 18 | 13.87 | 6.38 | 13.61 | 3 |
| 19 | 14.13 | 6.26 | 95.69 | 1 |
| 20 | 14.52 | 6.09 | 51.73 | 2 |
| 21 | 15.06 | 5.88 | 32.44 | 3 |
| 22 | 15.98 | 5.54 | 37.62 | 2 |
| 23 | 16.27 | 5.44 | 13.97 | 3 |
| 24 | 16.54 | 5.36 | 30.28 | 3 |
| 25 | 16.81 | 5.27 | 17.91 | 3 |
| 26 | 17.10 | 5.18 | 40.75 | 2 |
| 27 | 17.58 | 5.04 | 19.98 | 3 |
| 28 | 17.76 | 4.99 | 33.68 | 3 |
| 29 | 17.94 | 4.94 | 97.66 | 1 |
| 30 | 18.54 | 4.78 | 10.97 | 3 |
| 31 | 18.72 | 4.74 | 21.16 | 3 |
| 32 | 19.27 | 4.60 | 12.94 | 3 |
| 33 | 19.98 | 4.44 | 36.04 | 2 |
| 34 | 20.25 | 4.38 | 17.25 | 3 |
| 35 | 20.55 | 4.32 | 69.63 | 2 |
| 36 | 20.77 | 4.27 | 24.56 | 3 |
| 37 | 21.15 | 4.20 | 23.39 | 3 |
| 38 | 21.22 | 4.18 | 25.15 | 3 |
| 39 | 21.43 | 4.14 | 20.21 | 3 |
| 40 | 21.88 | 4.06 | 22.98 | 3 |
| 41 | 22.06 | 4.03 | 39.66 | 2 |
| 42 | 22.12 | 4.02 | 39.47 | 2 |
| 43 | 22.55 | 3.94 | 44.31 | 2 |
| 44 | 22.82 | 3.89 | 29.36 | 3 |
| 45 | 22.95 | 3.87 | 28.35 | 3 |
| 46 | 23.07 | 3.85 | 37.80 | 2 |
| 47 | 23.13 | 3.84 | 38.97 | 2 |
| 48 | 23.34 | 3.81 | 46.12 | 2 |
| 49 | 24.33 | 3.65 | 10.13 | 3 |
| 50 | 26.04 | 3.42 | 13.06 | 3 |
| 51 | 26.33 | 3.38 | 13.92 | 3 |
| 52 | 26.82 | 3.32 | 17.29 | 3 |
| 53 | 27.18 | 3.28 | 25.74 | 3 |
| 54 | 27.57 | 3.23 | 10.37 | 3 |
| 55 | 27.97 | 3.19 | 17.69 | 3 |
| 56 | 28.18 | 3.16 | 25.57 | 3 |
| 57 | 28.44 | 3.14 | 10.66 | 3 |
| 58 | 28.65 | 3.11 | 10.06 | 3 |
| 59 | 28.76 | 3.10 | 16.39 | 3 |
| 60 | 29.28 | 3.05 | 15.51 | 3 |
| 61 | 29.71 | 3.00 | 15.49 | 3 |
| 62 | 29.77 | 3.00 | 14.78 | 3 |
| 63 | 29.89 | 2.99 | 11.65 | 3 |

In a further aspect, the PXRD peak locations displayed in Table 4 and/or FIG. 4 most characteristic of crystalline form Type 8 of sugammadex can be selected and grouped as “diagnostic peak sets” to conveniently distinguish this crystalline form from others. Selections of such characteristic peaks are set out in Table 4 in the column labeled Diagnostic Peak Set.

Thus, in another aspect, there is provided a crystalline form Type 8 of sugammadex characterized by a powder x-ray diffraction pattern comprising each of the 2-theta values listed in Diagnostic Peak Set 1 in Table 4, +/−0.2° 2-theta.

In another aspect, there is provided a crystalline form Type 8 of sugammadex characterized by a powder x-ray diffraction pattern comprising each of the 2-theta values listed in Diagnostic Peak Set 2 in Table 4, +/−0.2° 2-theta.

In another aspect, there is provided a crystalline form Type 8 of sugammadex characterized by a powder x-ray diffraction pattern comprising each of the 2-theta values listed in Diagnostic Peak Set 3 in Table 4, +/−0.2° 2-theta.

In another aspect, there is provided a crystalline form Type 8 of sugammadex characterized by a powder x-ray diffraction pattern comprising each of the 2-theta values listed in Diagnostic Peak Set 1 and Diagnostic Peak Set 2 in Table 4, +/−0.2° 2-theta.

In another aspect, there is provided a crystalline form Type 8 of sugammadex characterized by a powder x-ray diffraction pattern comprising each of the 2-theta values listed in Diagnostic Peak Set 1 and Diagnostic Peak Set 2 and Diagnostic Peak Set 3 in Table 4, +/−0.2° 2-theta.

### Crystalline Form Type 9 of Sugammadex

Type 9 appeared as an intermediate and metastable form in a process conducted to generate Type 3. Crystalline form Type 9 of sugammadex was prepared as follows: A clear solution of 30 g of sugammadex in 90 ml purified water was prepared. The solution was agitated at 200 rpm for 5 min at ambient conditions, heated to 40° C. over 10 minutes, and aged for an additional 10 minutes. Subsequently, several methanol addition and aging steps were conducted as follows: 350 mL of methanol were added linearly over 70 min, producing a slurry. The slurry was aged for 60 minutes, and then 20 ml of methanol was added linearly over 5 minutes followed by the addition of 80 ml of methanol linearly over 30 minutes. The slurry was then aged for 60 minutes until the methanol:water ratio reached 5:1. A wet cake sample was produced by centrifuging an aliquot of the slurry to a wet paste. PXRD analysis of the wet cake produced the Type 9 pattern.

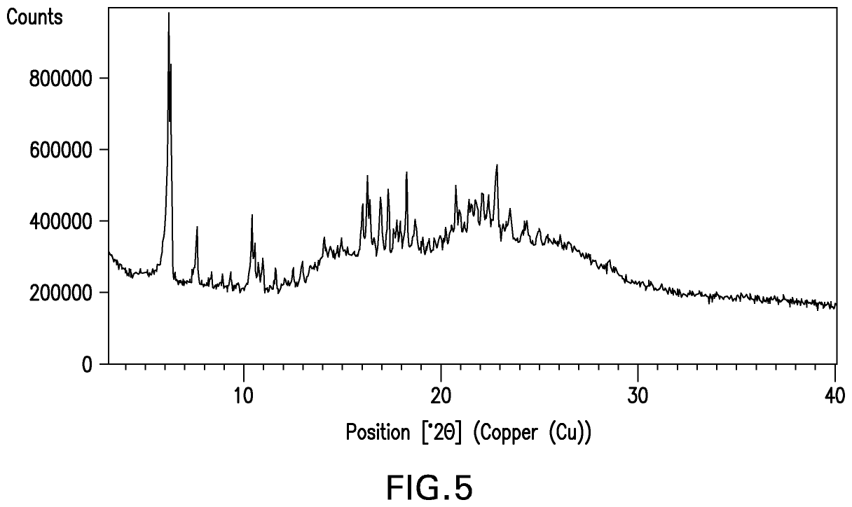

A PXRD pattern of crystalline form Type 9 of sugammadex generated using the equipment and procedures described above is displayed in FIG. 5. Thus, in another aspect, there is provided a crystalline form Type 9 of sugammadex characterized by a powder x-ray diffraction pattern substantially as shown in FIG. 5.

The intensity of the peaks (y-axis is in counts per second) were plotted versus the 2 theta angle (x-axis is in degrees 2 theta). In addition, the data were plotted with detector counts normalized for the collection time per step versus the 2 theta angle. Peak locations (on the 2 theta x-axis) consistent with these profiles are displayed in Table 5 (+/−0.2° 2 theta). The locations of these PXRD peaks are characteristic of the crystalline form Type 9 of sugammadex. Thus, in another aspect, crystalline form Type 9 of sugammadex is characterized by a powder x-ray diffraction pattern having each of the peak positions listed in Table 5, +/−0.2° 2-theta.

TABLE 5
Diffraction peaks and corresponding d-spacings for crystalline
form Type 9 of sugammadex

| | | | | Preferred |
|---|---|---|---|---|
| Peak | Pos. [° Two | | | Diagnostic Peak |
| Number | Theta] | d-spacing [Å] | Rel. Int. [%] | Set |
| 1 | 6.00 | 14.71 | 100.00 | 1 |
| 2 | 6.11 | 14.45 | 80.22 | 1 |
| 3 | 7.42 | 11.90 | 23.24 | 2 |
| 4 | 8.18 | 10.80 | 5.55 | 3 |
| 5 | 8.74 | 10.11 | 3.93 | 3 |
| 6 | 9.13 | 9.67 | 4.75 | 3 |
| 7 | 10.23 | 8.64 | 26.29 | 1 |
| 8 | 10.36 | 8.53 | 15.37 | 2 |
| 9 | 10.56 | 8.37 | 10.91 | 2 |
| 10 | 10.77 | 8.21 | 12.32 | 2 |
| 11 | 11.42 | 7.74 | 7.20 | 3 |
| 12 | 11.91 | 7.43 | 3.79 | 3 |
| 13 | 12.32 | 7.18 | 5.69 | 3 |
| 14 | 12.79 | 6.92 | 6.97 | 3 |
| 15 | 13.20 | 6.70 | 3.77 | 3 |
| 16 | 13.87 | 6.38 | 10.50 | 2 |
| 17 | 14.03 | 6.31 | 3.93 | 3 |
| 18 | 14.14 | 6.26 | 3.86 | 3 |
| 19 | 14.25 | 6.21 | 5.40 | 3 |
| 20 | 14.77 | 5.99 | 6.59 | 3 |
| 21 | 15.83 | 5.59 | 18.88 | 2 |
| 22 | 16.08 | 5.51 | 27.95 | 1 |
| 23 | 16.21 | 5.46 | 20.48 | 2 |
| 24 | 16.41 | 5.40 | 6.55 | 3 |
| 25 | 16.75 | 5.29 | 21.22 | 2 |
| 26 | 17.13 | 5.17 | 24.11 | 2 |
| 27 | 17.43 | 5.08 | 9.91 | 3 |
| 28 | 17.58 | 5.04 | 14.03 | 2 |
| 29 | 17.76 | 4.99 | 12.73 | 2 |
| 30 | 18.06 | 4.91 | 27.89 | 1 |
| 31 | 18.48 | 4.80 | 11.19 | 2 |
| 32 | 18.55 | 4.78 | 12.79 | 2 |
| 33 | 18.89 | 4.69 | 4.00 | 3 |
| 34 | 19.18 | 4.62 | 3.71 | 3 |
| 35 | 19.81 | 4.48 | 3.70 | 3 |
| 36 | 20.06 | 4.42 | 5.22 | 3 |
| 37 | 20.58 | 4.31 | 20.53 | 2 |
| 38 | 20.78 | 4.27 | 12.41 | 2 |
| 39 | 20.94 | 4.24 | 4.93 | 3 |
| 40 | 21.03 | 4.22 | 4.57 | 3 |
| 41 | 21.21 | 4.19 | 12.80 | 2 |
| 42 | 21.36 | 4.16 | 14.17 | 2 |
| 43 | 21.57 | 4.12 | 15.01 | 2 |
| 44 | 21.91 | 4.05 | 18.02 | 2 |
| 45 | 22.24 | 3.99 | 14.04 | 2 |
| 46 | 22.63 | 3.93 | 26.64 | 1 |
| 47 | 23.01 | 3.86 | 4.01 | 3 |
| 48 | 23.10 | 3.85 | 5.10 | 3 |
| 49 | 23.34 | 3.81 | 10.63 | 2 |
| 50 | 24.16 | 3.68 | 7.18 | 3 |
| 51 | 24.81 | 3.59 | 6.06 | 3 |
| 52 | 25.24 | 3.53 | 4.01 | 3 |
| 53 | 25.89 | 3.44 | 3.99 | 3 |
| 54 | 26.31 | 3.39 | 3.07 | 3 |
| 55 | 26.41 | 3.37 | 3.20 | 3 |
| 56 | 27.33 | 3.26 | 3.47 | 3 |
| 57 | 27.58 | 3.23 | 3.66 | 3 |
| 58 | 27.71 | 3.22 | 4.02 | 3 |
| 59 | 27.94 | 3.19 | 4.89 | 3 |
| 60 | 28.12 | 3.17 | 5.33 | 3 |
| 61 | 28.36 | 3.14 | 7.60 | 3 |
| 62 | 28.59 | 3.12 | 5.27 | 3 |
| 63 | 28.72 | 3.11 | 5.89 | 3 |

In a further aspect, the PXRD peak locations displayed in Table 5 and/or FIG. 5 most characteristic of crystalline form Type 9 of sugammadex can be selected and grouped as “diagnostic peak sets” to conveniently distinguish this crystalline form from others. Selections of such characteristic peaks are set out in Table 5 in the column labeled Diagnostic Peak Set.

Thus, in another aspect, there is provided a crystalline form Type 9 of sugammadex characterized by a powder x-ray diffraction pattern comprising each of the 2-theta values listed in Diagnostic Peak Set 1 in Table 5, +/−0.2° 2-theta.

In another aspect, there is provided a crystalline form Type 9 of sugammadex characterized by a powder x-ray diffraction pattern comprising each of the 2-theta values listed in Diagnostic Peak Set 2 in Table 5, +/−0.2° 2-theta.

In another aspect, there is provided a crystalline form Type 9 of sugammadex characterized by a powder x-ray diffraction pattern comprising each of the 2-theta values listed in Diagnostic Peak Set 3 in Table 5, +/−0.2° 2-theta.

In another aspect, there is provided a crystalline form Type 9 of sugammadex characterized by a powder x-ray diffraction pattern comprising each of the 2-theta values listed in Diagnostic Peak Set 1 and Diagnostic Peak Set 2 in Table 5, +/−0.2° 2-theta.

In another aspect, there is provided a crystalline form Type 9 of sugammadex characterized by a powder x-ray diffraction pattern comprising each of the 2-theta values listed in Diagnostic Peak Set 1 and Diagnostic Peak Set 2 and Diagnostic Peak Set 3 in Table 5, +/−0.2° 2-theta.

### Additional Characteristics

Among the crystalline forms of sugammadex described herein, crystalline form Type 1 of sugammadex have been found to be particularly amenable to purification after isolation from a plant crystallization process using a methanol/water solvent system, and thus more readily able to meet residual solvent specifications. As such, crystalline form Type 1 of sugammadex is particularly useful in the preparation drug product (described below). Crystalline forms Type 2 and Type 3 of sugammadex typically isolate as large particles which are relatively more difficult than crystalline form Type 1 to dry and are therefore less preferred than Type 1. Nevertheless, it has also been found that crystalline forms Type 2 and Type 3 of sugammadex, when present, may advantageously be recrystallized into Type 1. Type 8 and Type 9 have been found to be metastable crystalline forms and can be encountered at certain stages of a typical crystallization. As such, isolation of Types 8 and 9 at the end of a typical plant crystallization is not observed.

### Preparation Of Drug Product

Crystallized Type 1 form of sugammadex was prepared as described above, then isolated as a wet cake and then dried under vacuum to obtain a powder meeting purity and residual solvent specifications. The drug product was produced by a formulation and filling process. The dried sugammadex powder is dissolved in water for injection and the pH adjusted to 7.5. The resulting solution was then filtered and filled into vials, stoppered and capped. The bulk drug product was then terminally sterilized and stored for use.

1. The crystalline form Type 1 of sugammadex characterized by a powder x-ray diffraction pattern obtained using Cu K alpha radiation with at least peaks at diffraction angles degrees 2 theta (+/−0.2°) of:
6.04
16.01
17.05
21.83
22.50
23.31.

2. The crystalline form Type 1 of sugammadex according to claim 1, further characterized by a powder x-ray diffraction pattern with at least peaks at diffraction angles degrees 2 theta (+/−0.2°) of:
6.04
7.80
10.42
14.72
16.01
16.79
17.05
17.59
17.86
18.12
18.91
21.01
21.83
22.24
22.50
22.90
23.31
24.30
24.92
28.47.

3. The crystalline form Type 1 of sugammadex according to claim 1, further characterized by a powder x-ray diffraction pattern with at least peaks at diffraction angles degrees 2 theta (+/−0.2°) of:
6.04
7.80
9.39
9.83
10.42
11.14
11.57
11.99
12.36
13.33
14.09
14.40
14.72
15.05
15.31
16.01
16.79
17.05
17.35
17.59
17.86
18.12
18.42
18.91
20.09
20.30
20.51
21.01
21.83
22.24
22.50
22.90
23.31
23.55
24.30
24.73
24.92
25.43
25.81
25.98
26.30
26.61
27.05
27.25
27.64
27.95
28.47
28.95
29.17
29.53
29.76
29.94.

4. A crystalline form Type 2 of sugammadex characterized by a powder x-ray diffraction pattern obtained using Cu K alpha radiation with at least peaks at diffraction angles degrees 2 theta (+/−0.2°) of:
5.72
7.07
8.98
17.44
18.28
22.37
22.55.

5. The crystalline form Type 2 of sugammadex according to claim 4, further characterized by a powder x-ray diffraction pattern with at least peaks at diffraction angles degrees 2 theta (+/−0.2°) of:
5.72
7.07
8.98
15.31
15.49
16.81
17.22
17.44
18.28
20.95
22.37
22.55
22.91
23.27
23.84
25.37
28.56
28.88
29.26.

6. The crystalline form Type 2 of sugammadex according to claim 4, further characterized by a powder x-ray diffraction pattern with at least peaks at diffraction angles degrees 2 theta (+/−0.2°) of:
5.72
6.94
7.07
7.37
7.73
8.18
8.38
8.98
9.64
10.13
10.22
11.44
11.51
12.41
13.55
13.82
13.93
14.29
14.68
15.31
15.49
15.76
15.98
16.37
16.81
17.22
17.44
18.28
18.59
18.70
19.40
19.63
19.83
20.32
20.46
20.62
20.95
21.31
21.54
21.74
21.97
22.37
22.55
22.91
23.27
23.56
23.84
24.78
25.37
26.04
26.38
26.87
27.07
27.34
27.52
28.07
28.29
28.56
28.88
29.26
29.86.

7. The crystalline form Type 3 of sugammadex characterized by a powder x-ray diffraction pattern obtained using Cu K alpha radiation with at least peaks at diffraction angles degrees 2 theta (+/−0.2°) of:
5.60
16.80
17.81
18.10
22.38.

8. The crystalline form Type 3 of sugammadex according to claim 7, further characterized by a powder x-ray diffraction pattern with at least peaks at diffraction angles degrees 2 theta (+/−0.2°) of:
5.60
11.47
14.23
16.80
17.81
18.10
18.71
21.82
22.38
22.74
23.48
24.45
25.15
26.23
31.27
31.43
31.79
33.49
33.94
34.33.

9. The crystalline form Type 3 of sugammadex according to claim 8, further characterized by a powder x-ray diffraction pattern with at least peaks at diffraction angles degrees 2 theta (+/−0.2°) of:
5.60
7.75
7.89
7.96
8.41
8.72
8.84
11.13
11.47
11.83
13.99
14.23
15.83
16.80
17.36
17.81
18.10
18.38
18.71
18.87
19.91
20.44
20.92
21.57
21.82
22.38
22.58
22.74
22.96
23.12
23.48
23.82
24.04
24.14
24.45
24.63
24.77
25.15
25.37
25.58
25.78
25.98
26.23
26.70
27.17
27.46
28.10
28.79
29.24
29.76
30.12
30.89
31.27
31.43
31.79
32.08
32.28
32.68
33.00
33.49
33.94
34.33.

10. The crystalline form Type 8 of sugammadex characterized by a powder x-ray diffraction pattern obtained using Cu K alpha radiation with at least peaks at diffraction angles degrees 2 theta (+/−0.2°) of:
6.19
8.85
9.66
14.13
17.94.

11. The crystalline form Type 8 of sugammadex according to claim 10, further characterized by a powder x-ray diffraction pattern with at least peaks at diffraction angles degrees 2 theta (+/−0.2°) of:
4.82
6.19
8.85
9.66
10.89
12.00
13.48
14.13
14.52
15.98
17.10
17.94
19.98
20.55
22.06
22.12
22.55
23.07
23.13
23.34.

12. The crystalline form Type 8 of sugammadex according to claim 11, further characterized by a powder x-ray diffraction pattern with at least peaks at diffraction angles degrees 2 theta (+/−0.2°) of:
4.48
4.82
6.19
6.73
7.09
7.97
8.85
9.54
9.66
10.27
10.36
10.89
12.00
12.38
13.08
13.48
13.64
13.87
14.13
14.52
15.06
15.98
16.27
16.54
16.81
17.10
17.58
17.76
17.94
18.54
18.72
19.27
19.98
20.25
20.55
20.77
21.15
21.22
21.43
21.88
22.06
22.12
22.55
22.82
22.95
23.07
23.13
23.34
24.33
26.04
26.33
26.82
27.18
27.57
27.97
28.18
28.44
28.65
28.76
29.28
29.71
29.77
29.89.

13. The crystalline form Type 9 of sugammadex characterized by a powder x-ray diffraction pattern obtained using Cu K alpha radiation with at least peaks at diffraction angles degrees 2 theta (+/−0.2°) of:
6.00
6.11
10.23
16.08
18.06
22.63.

14. The crystalline form Type 9 of sugammadex according to claim 13, further characterized by a powder x-ray diffraction pattern with at least peaks at diffraction angles degrees 2 theta (+/−0.2°) of:
6.00
6.11
7.42
10.23
10.36
10.56
10.77
13.87
15.83
16.08
16.21
16.75
17.13
17.58
17.76
18.06
18.48
18.55
20.58
20.78
21.21
21.36
21.57
21.91
22.24
22.63
23.34.

15. The crystalline form Type 9 of sugammadex according to claim 14, further characterized by a powder x-ray diffraction pattern with at least peaks at diffraction angles degrees 2 theta (+/−0.2°) of:
6.00
6.11
7.42
8.18
8.74
9.13
10.23
10.36
10.56
10.77
11.42
11.91
12.32
12.79
13.20
13.87
14.03
14.14
14.25
14.77
15.83
16.08
16.21
16.41
16.75
17.13
17.43
17.58
17.76
18.06
18.48
18.55
18.89
19.18
19.81
20.06
20.58
20.78
20.94
21.03
21.21
21.36
21.57
